(19) United States
(12) Patent Application Publication
(10) Pub. No.: US 2005/0191754 A1
(43) Pub. Date: Sep. 1, 2005

(54) Process for checking the processing of the exhaust gases of a heat engine and vehicle with a heat engine using this process

(76) Inventors: Arnaud Audouin, Paris (FR); Alain Sassi, Montigny Le Bretonneux (FR)

Correspondence Address: RATNERPRESTIA, P O BOX 980, VALLEY FORGE, PA, 19482-0980, US

(21) Appl. No.: 11/068,001

(22) Filed: Feb. 28, 2005

(30) Foreign Application Priority Data
Feb. 27, 2004 (FR) 04/50385

(51) Int. Cl. G01N 33/00

(52) U.S. Cl. 436/116

(57) ABSTRACT

The present invention pertains to a process for checking the processing of the exhaust gases of a heat engine, the nitrogen oxides (NOx) contained in these gases being stored and then reduced in a trap modeled in such a way that the reduction of these nitrogen oxides (NOx) is actuated when the storage capacity of the trap, determined according to the model, reaches a threshold (Mₜₕᵣₑₛₕ). According to the present invention, such a process is characterized in that the modeling of the trap is checked by measuring the quantity of nitrogen oxides (NOx) stored in this trap by means of a sensor located downstream of the trap, and by comparing this measurement to the quantity of nitrogen oxides (NOx) stored, determined according to the model, in order to correct the latter if the measured quantity is markedly different from the modeled quantity.

The present invention pertains to a process for checking the processing of exhaust gases of a heat engine and to a vehicle with a heat engine using this process, and in particular by means of a nitrogen oxide trap designed to reduce the level of nitrogen oxides present in the exhaust gases.

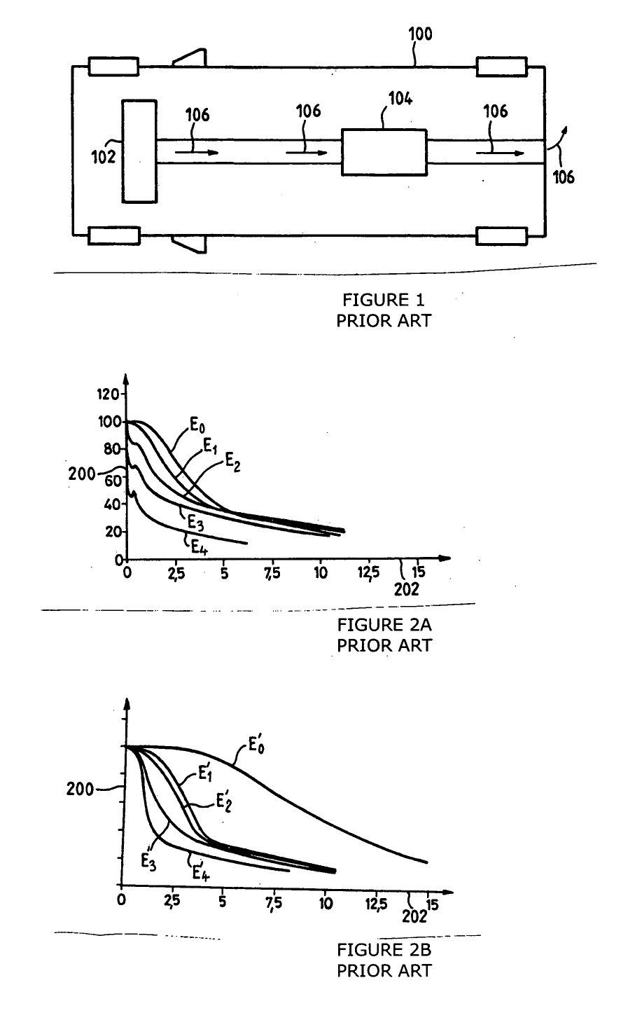

It is known to equip a vehicle 100 (FIG. 1) with a catalytic converter designed to process the exhaust gases 106 emitted by its engine 102, and this catalytic converter may be of the nitrogen oxide (NOx) trap 104 type.

In this case, this trap 104 consists of materials with an affinity for nitrogen oxides, in order, at first, to retain the latter when the gases 106 pass through the trap 104 and subsequently to make it possible to reduce their nitrogen (N₂) content. In fact, such a trap 104 alternates between two modes of operation, which are characteristic of the nitrogen oxide trap and are described in detail below:

A first mode of operation corresponds to a storage of the nitrogen oxides, during which the trap 104 collects the latter in the exhaust gases 106.

This mode corresponds to a so-called “lean” operation of the engine such that the oxygen is present in excess compared to the fuel. In this case, the richness r of the mixture, which is equal to the ratio of the quantity of fuel to the quantity of oxygen, is less than one.

During this first mode of operation, the storage of nitrogen oxides is limited by the storage capacity of the trap 104, which can be defined as the maximum mass Mₐₓ of nitrogen oxides that this trap 104 can collect.

By considering the entering NOx mass Mₑₙₜ(t) and the exiting NOx mass Mₑₓᵢₜ(t) at a given instant of the nitrogen oxide trap 104, the storage efficiency E(t) of this trap 104 can be defined as the difference between the nitrogen oxide entering mass Mₑₙₜ(t) and exiting mass Mₑₓᵢₜ(t) divided by the nitrogen oxide entering mass Mₑₙₜ(t).

Such a definition thus corresponds to the following formula (1):

$$E ( t ) = \frac{{M}_{ent} ( t ) - {M}_{exit} ( t )}{{M}_{ent} ( t )} \qquad ( 1 )$$

This formula (1) reflects the decrease in the efficiency E(t) of a nitrogen oxide trap as soon as the nitrogen oxide mass stored tends toward the maximum nitrogen oxide mass Mₐₓ(T) that can be stored because, in this case, the nitrogen oxide exiting mass Mₑₓᵢₜ(t) tends toward the nitrogen oxide entering mass Mₑₙₜ(t).

This decrease is empirically measurable as is shown in FIGS. 2a and 2b, which plot the efficiency E(t) (ordinate 200, in percentage) of the nitrogen oxide trap 104 against the nitrogen oxide mass (abscissa 202, in grams) stored in this trap 104.

Furthermore, FIG. 2a also shows that the efficiency E(t) of the nitrogen oxide trap also decreases when the quantity of sulfur (S) collected by the trap increases in the latter, this decrease being due to a lowering of the storage capacity of the trap.

In fact, the efficiencies E₀, E₁, E₂, E₃ and E₄, measured for traps having a sulfur content close to 0, 1, 2, 3 and 4 grams per liter, respectively, are decreasing for a same stored quantity of nitrogen oxides.

This is why it is necessary to carry out operations for the removal of sulfur (desulfurization) at regular intervals in order to restore its storage capacity.

However, such operations of removing sulfur have the drawback of irreversibly reducing the storage capacity, and therefore the efficiency, of the trap on the long term as shown below by means of FIG. 2b.

Thus, the higher this number of cycles, the lower are the efficiencies E′₀, E′₁, E′₂, E′₃ and E′₄ measured for traps having undergone increasing numbers of sulfur storage/removal cycles (0, 5, 10, 18 and 30, respectively).

In fact, these sulfur removal operations subject the trap to high temperatures (greater than 600° C.) for a period generally ranging from 4 to 20 minutes, which brings about the degradations, called thermal aging, of the catalytic phase of the trap.

This is why it is known to check the frequency of the sulfur removal of a trap by determining the quantity of sulfur received by the latter from the consumption of the vehicle and from a sulfur content attributed to the fuel.

A second mode of operation of the trap 104 corresponds to the nitrogen (N₂) reduction of the nitrogen oxides collected by this trap, the latter reacting with the reducers (HC: hydrocarbons, CO: carbon monoxide and H₂: hydrogen) supplied by the engine 102 via the exhaust gases 106.

For this, the quantity of hydrocarbons supplied to the trap 104 is increased by means of a so-called “rich” operation of the engine 102, the quantity of fuel introduced in the engine being greater than the quantity of oxygen in relation to stoichiometric conditions, and the richness r of the mixture is greater than 1.

This mode of removal requires a good determination of the quantity of nitrogen oxides present in the trap 104 in order to actuate the engine in such a way that it supplies the optimal ratio, called λ, between the quantity of oxygen (oxidant) and the quantity of reducers (HC, CO and H₂) in the exhaust gases.

Actually, if there is a lack of oxygen compared to the reducers, the latter are emitted into the environment, while the reduction of nitrogen oxides would be incomplete due to a lack of reducers if an excess of oxygen was present.

This determination is currently made by means of an operation model of the trap 104, which aims at predetermining the storage capacity of the latter as a function, for example, of the number of times nitrogen oxides or sulfur have been removed, in order to optimally actuate new removal operations.

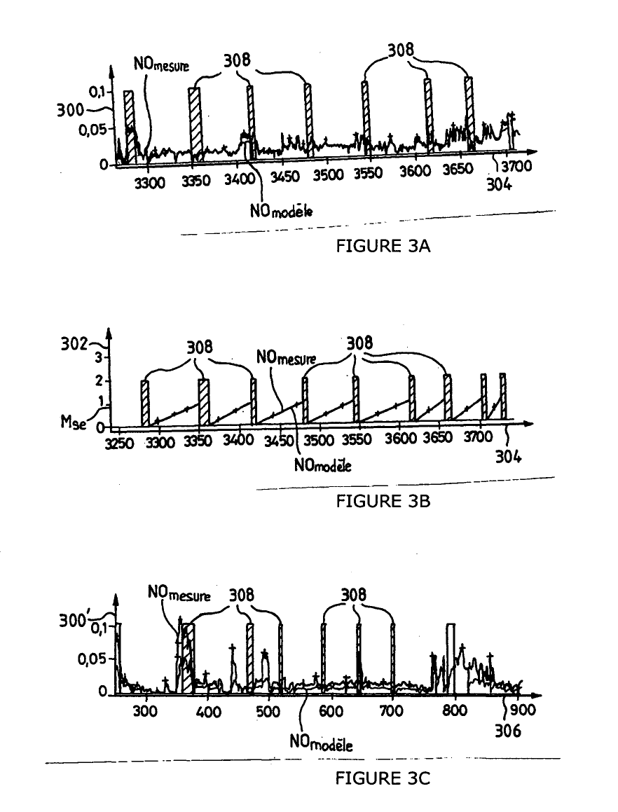

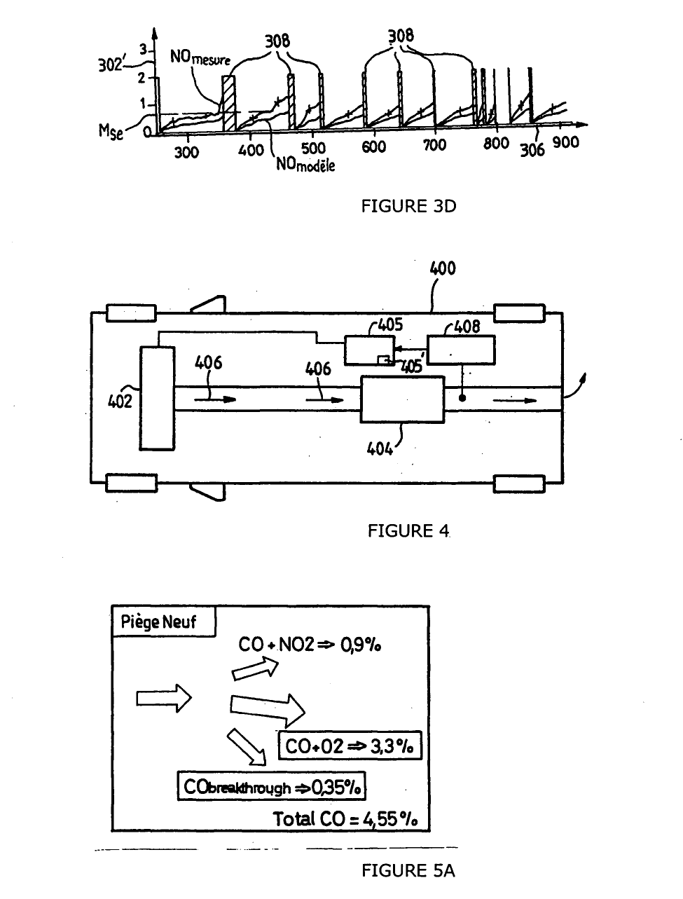

The present invention is a result of the observation that, during its operation, the variation of the storage capacity of a nitrogen oxide trap, described above by means of FIGS. 2a and 2b, may be such that the operation of the trap differs significantly from its modeling, as described below by means of FIGS. 3a, 3b, 3c and 3d.

The presence of such a difference, or drift, of the trap prevents its optimal management, and in particular as regards actuated removals, in such a way that the nitrogen oxide level in the exhaust gases may increase, during the operation of the engine, beyond thresholds previously observed.

The present invention is also a result of the observation that such a drift is unforeseeable given that the sulfur content of the fuel used by a vehicle is variable, for example, from one country to the next.

This is why the present invention aims to provide a process for checking the operation of a catalytic converter provided with a nitrogen oxide trap.

More precisely, the present invention pertains to a process for checking the processing of the exhaust gases of a heat engine, the nitrogen oxides contained in these gases being stored, then reduced in a modeled trap in such a way that the reduction of these nitrogen oxides is actuated when the storage capacity of the trap, determined according to the model, reaches a threshold, characterized in that the modeling of the trap is checked by measuring the quantity of nitrogen oxides stored in this trap by means of a NOx sensor located downstream of the trap, and by comparing this measurement to the stored quantity of nitrogen oxides, determined according to the model, in order to correct the latter if the measured quantity of nitrogen oxides is markedly different from the quantity predetermined according to the model.

Such a process, in which the quantity of nitrogen oxides stored in the trap is measured directly, has the advantage of making it possible to correct the nitrogen oxide storage model of the trap in question as this trap wears out, in such a way that its actuation corresponds to its real storage capacity. Thus, the removals of nitrogen oxides or sulfur from storage can be actuated optimally, minimizing the quantity and the duration of these removals, the wear and tear of the trap and the quantity of hydrocarbons, in particular carbon monoxide, emitted outside the vehicle.

In one embodiment, the measurement of the quantity of nitrogen oxides stored in the trap is determined by performing an average of different measurements carried out on different nitrogen oxide storage and reduction cycles.

According to one embodiment, the quantity of nitrogen oxides stored in the trap is measured by means of a nitrogen oxide sensor supplying a signal, whose level is proportional to the quantity of nitrogen oxides exiting the trap.

In one embodiment, the quantities of nitrogen oxides, measured or modeled, are considered to be masses of nitrogen oxides, for example, in grams.

According to one embodiment, the model is modified by determining a new storage capacity of the trap as the product of the previous storage capacity by the ratio of the stored quantity of nitrogen oxides according to the measurement to the stored quantity of nitrogen oxides according to the model.

In one embodiment, a removal of sulfur is actuated when the ratio of the measured quantity of nitrogen oxides to the modeled quantity of nitrogen oxides exceeds a predetermined threshold.

According to one embodiment, with a sensor determining the oxygen content of the exhaust gases after their being processed by the trap, the level of the signal emitted by this sensor is used for determining an oxidation capacity of the trap.

In one embodiment, the level of the signal depends on the quantity of hydrocarbons present in the exhaust gases.

According to one embodiment, the sensor is a nitrogen oxide (NOx) sensor, also delivering information of the λ type sensor.

The present invention also pertains to a vehicle provided with means for checking the processing of the exhaust gases of a heat engine, the nitrogen oxides contained in these gases being stored and then reduced in a trap modeled in such a way that the reduction of these nitrogen oxides is actuated when the storage capacity of the trap, determined according to the model, reaches a threshold, characterized in that it comprises means for checking the modeling of the trap according to a process in conformity with one of the above embodiments.

Other features and advantages of the present invention shall become apparent with the description provided above, by way of illustrative and nonlimiting example, with reference to the attached figures, in which:

FIG. 1, already described, schematically shows a prior-art processing of exhaust gases of a heat engine by a nitrogen oxide trap,

FIGS. 2a and 2b, already described, show efficiency variations of a nitrogen oxide trap,

FIGS. 3a, 3b, 3c and 3d illustrate the differences between the real operation and the predetermined or modeled operation of a nitrogen oxide trap,

FIG. 4 schematically shows a processing in accordance with the present invention of the exhaust gases of a heat engine by a nitrogen oxide trap,

FIGS. 5a and 5b show the different reactions of the carbon monoxide supplied by an engine in rich mode for a new nitrogen oxide trap and for a used trap,

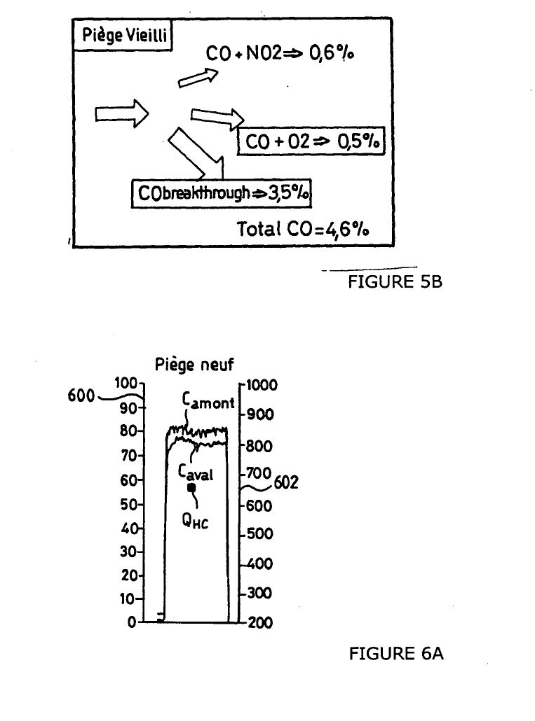

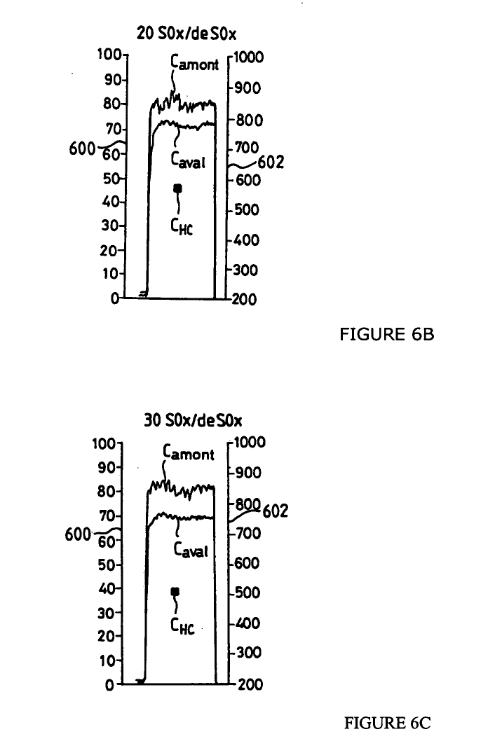

FIGS. 6a, 6b and 6c show variations of a signal emitted by a sensor measuring the oxygen λ ratio in the exhaust gases upstream of a nitrogen oxide trap.

As indicated above, the operation drift of a nitrogen oxide trap may reach high values as shown below by means of FIGS. 3a and 3b, pertaining to a new nitrogen oxide trap, and 3c and 3d, pertaining to the same trap after a significant use of the latter, for example, after 10 to 20 sulfur storage/removal sequences.

FIG. 3a shows instantaneous measurements related to masses (ordinate 300, in grams per second) of nitrogen oxides collected by the trap in question, while FIG. 3b shows the course of the total nitrogen oxide mass stored in this trap (ordinate 302, in grams) according to a same chronology (axis 304, in seconds).

In addition, the periods 308 of nitrogen oxide removal, triggered when the stored nitrogen oxide mass exceeds a threshold value Mₜₕᵣₑₛₕ, are also shown.

When the trap in question is new, the measured masses (NOₘₑₐₛᵤᵣₑₘₑₙₜ curve) correspond to the masses predetermined by an operating model (NOx_{model} curve) of the trap.

However, after a significant use of the trap, the measured masses (NOxₘₑₐₛᵤᵣₑₘₑₙₜ curve) are greatly different—up to a difference of 50%—from the masses predetermined (NOx_{model} curve) by the operating model of the trap, as shown by means of FIGS. 3c and 3d, which show, respectively, measurements (NOₘₑₐₛᵤᵣₑₘₑₙₜ curve) related to the mass (ordinate 300′, in grams per second) of nitrogen oxides collected by this used trap and to the mass of nitrogen oxides stored (ordinate 302′, in grams) in this used trap, according to a same chronology (axis 306, in seconds), as well as the predetermined measurements (NOx_{model} curve).

This is why, according to the present invention, the operating model of the trap is modified, in particular for determining the frequency of optimally removing NOx and sulfur from storage.

For this purpose, a vehicle 400 (FIG. 4) according to the present invention is provided with a trap 404 processing the exhaust gases 406 emitted by its engine 402 and with a processor 405 provided with means 405′ designed to modify the predetermined operating model of the trap 404 as a function of the measured variations of the storage capacity of this trap 404.

For this, this processor 405 determines the quantity of nitrogen oxides stored in the trap, for example, in the form of the mass collected by the trap at an instant or over a given period, and this measurement is determined from the measurements of a nitrogen oxide sensor 408 located downstream of the trap 404, the latter sensor supplying different signals, i.e.:

A first “λ ON/OFF” electric signal, whose voltage is zero when the engine operates in lean mode, or maximum when the engine operates in rich mode,

A second “linear λ” electric signal, whose voltage is proportional to the oxygen X ratio in the exhaust gases, and

A third “NOx” electric signal, whose voltage is proportional to the nitrogen oxide concentration in the trap 404 when the engine operates in lean mode.

At this stage, it should be recalled that a nitrogen oxide sensor 408 provides a measurement of the nitrogen oxides in the trap 404 using the gas partial pressure difference between a reference cell and the exhaust gases.

From this third signal, the processor 405 can therefore determine, at first, the quantity of nitrogen oxides stored in the trap 404 and then, subsequently, the difference between the measured quantity and the predetermined quantity of nitrogen oxide stored in the trap.

In this preferred embodiment of the present invention, the processor 405 carries out such a determination by performing averages of the quantities measured during the operation of the trap according to the storage mode and according to the mode of removal of the trap, for example, by considering 50 to 100 measurements so as to define a ratio of the measured averages to the predetermined averages corresponding to the model.

Consequently, if the model is adapted to the aging of the trap, this ratio is kept practically equal to 1, while, if the storage capacity of the trap varies markedly from the model, this ratio is not equal to 1. In this example, the accepted ratio runs up to 2 before the model of the trap is modified.

In the latter case, the processor 405 can thus actuate the removal of sulfur from storage, if the drift of the trap 404 is attributable to a poisoning of the trap with sulfur, or can modify the storage model used in order to adapt it to a new storage capacity if this drift is attributable to the wear and tear of the trap.

A second aspect of the present invention, which can be used independently, is a result of the observation that the oxidation capacity of a catalytic converter varies greatly during the operation of the nitrogen oxide trap, as shown below by means of FIGS. 5a and 5b.

The ratios, in which the carbon monoxide (CO) emitted by a heat engine processed and converted by a new nitrogen oxide trap, are indicated in FIG. 5a.

Thus, 20% of this carbon monoxide (CO) reacts with the nitrogen oxide (NOx), 70% of this carbon monoxide reacts with the oxygen (O₂) and less than 10% of this carbon monoxide is emitted into the environment, which represents an optimal operation of the engine/afterprocessing system.

As shown in FIG. 5b, the use of the same used nitrogen oxide trap, with the exhaust gases having the same oxygen λ ratio, gives rise to 13% of the carbon monoxide reacting with nitrogen oxides, 10% of this carbon monoxide reacting with the oxygen, while more than 75% of the carbon monoxide produced is emitted into the environment, which represents an insufficient operation of the trap as regards certain standards pertaining to exhaust gases.

In fact, the oxidation capacity of a trap decreases with the increase in its wear and tear in such a way that, for an optimal operation of this trap, the oxygen λ ratio in the exhaust gases should be increased in parallel with the increase in its wear and tear.

This is why, according to this aspect of the present invention, the oxidation capacity of a nitrogen oxide trap is evaluated regularly to adapt the oxygen λ ratio in the exhaust gases.

For this purpose, the processor 405 uses the variation of the maximum value of the “λ ON/OFF” electric signal supplied by the sensor 408 because, as described below by means of FIGS. 6a, 6b and 6c, the value of this signal is dependent on the quantity of hydrocarbons present in these gases for a given oxygen λ ratio of the exhaust gases.

These FIGS. 6a, 6b and 6c show the values of the voltage of the “λ ON/OFF” electric signal (ordinate 602, in mV), supplied by the sensor 408, as a function of the level of oxidation of the hydrocarbons (HC) measured experimentally for increasing wear levels of the nitrogen oxide trap in question.

This signal is generated upstream (Cᵤₚₛₜᵣₑₐₘ curve) and downstream (C_{downstream} curve) of the trap by a sensor (not shown), which makes it possible to observe that the value of this upstream voltage is independent of the quantity of hydrocarbons present in the exhaust gases.

However, it is observed that the voltage of the signal supplied by the sensor 408 downstream of the trap decreases as a function of the quantity of hydrocarbons present in the gases 406, and the more the Q_{HC} content (ordinate 600, in percentage of oxidized hydrocarbons) for converting hydrocarbons decreases, the greater is this quantity.

This variation of the signal emitted by the sensor 408 can be explained by recalling that the measurement of the oxygen content by a λ sensor downstream of the nitrogen oxide trap is carried out, theoretically, after oxidation of all the reducers contained in these exhaust gases.

However, the rate of diffusion of hydrocarbons within the trap 404 is lower than that of the other components, and in particular of oxygen, in such a way that, when the λ ratio is measured downstream of the trap 404, the higher the quantity of hydrocarbons is, the lower is this λ ratio.

Thus, it is possible to determine the hydrocarbon conversion rate of the trap as a function of the “λ ON/OFF” signal emitted by the sensor 408, and this conversion rate makes it possible to determine the oxidation capacity of the trap.

Consequently, by detecting a decrease in the oxidation capacity of the trap, a processor 405 according to the present invention can actuate an increase in the λ ratio in the exhaust gases in order to maintain the operation of the trap under optimal conditions.

1. A process for checking the processing of exhaust gases of a heat engine, wherein, nitrogen oxides (NOx) contained in these gases are stored and then reduced in a trap modeled in such a way that the reduction of the nitrogen oxides (NOx) is actuated when the storage capacity of the trap, determined according to the model, reaches a threshold, the process including the steps of:
checking the modeling of the trap by measuring a quantity of the nitrogen oxides (NOx) stored in the trap by means of a sensor located downstream of the trap,
comparing the measured quantity of nitrogen oxides to a modeled quantity of nitrogen oxides (NOx) stored, determined according to the model, and
correcting the model if the measured quantity of nitrogen oxides is different from the modeled quantity by more than a further threshold.

2. A process in accordance with claim 1, wherein the measurement of the quantity of nitrogen oxides (NOx) stored in the trap is determined by performing an average of different measurements carried out on respective different nitrogen oxide (NOx) storage and reduction cycles.

3. A process in accordance with claim 1 or 2, wherein the quantity of nitrogen oxides stored in the trap is measured by means of a nitrogen oxide (NOx) sensor supplying a signal the level of the signal being proportional to a quantity of nitrogen oxides (NOx) leaving the trap.

4. A process in accordance with claim 1 or 2, wherein the quantities of nitrogen oxides (NOx), measured or modeled, are masses of nitrogen oxides.

5. A process in accordance with claim 1 or 2, wheren the model is corrected by determining a new storage capacity of the trap as the product of the previous storage capacity and the ratio of the quantity of nitrogen oxides stored according to the measurement to the quantity of nitrogen oxides stored according to the model.

6. A process in accordance with claim 1 or 2, wherein removal of sulfur from storage is actuated when the ratio of the measured quantity of nitrogen oxides (NOx) to the modeled quantity of nitrogen oxides exceeds the further threshold.

7. A process in accordance with claim 1 or 2, further including the steps of determining oxygen (O₂) content of the exhaust gases after their processing by the trap and using the determined oxygen content to determine an oxidation capacity of the trap.

8. A process in accordance with claim 7, wherein the determined oxygen content depends on a quantity of hydrocarbons present in the exhaust gases.

9. A process in accordance with claim 7, characterized in that the sensor is a nitrogen oxide sensor, that also provides the determined oxygen content.

10. A vehicle provided with means for checking the processing of the exhaust gases of a heat engine, the nitrogen oxides (NOx) contained in these gases being stored and then reduced in a trap modeled in such a way that the reduction of these nitrogen oxides (NOx) is actuated when the storage capacity of the trap, determined according to the model, reaches a threshold (Mₜₕᵣₑₛₕ), wherein the means for checking the processing of the exhaust gasses comprises means for checking the modeling of the trap according to a process in accordance with claim 1 or claim 2.
